(19) United States
(12) Patent Application Publication
(10) Pub. No.: US 2024/0393305 A1
(43) Pub. Date: Nov. 28, 2024

(54) VANADIUM OXIDE-DOPED LASER-INDUCED GRAPHENE MULTI-PARAMETER SENSOR TO DECOUPLE SOIL NITROGEN LOSS AND TEMPERATURE

(71) Applicant: The Penn State Research Foundation, University Park, PA (US)

(72) Inventor: Huanyu Cheng, University Park, PA (US)

(21) Appl. No.: 18/666,900

(22) Filed: May 17, 2024

Related U.S. Application Data
(60) Provisional application No. 63/504,046, filed on May 24, 2023.

(51) Int. Cl. G01N 33/00; G01K 1/024; G01N 27/30

(52) U.S. Cl. CPC G01N 33/0037; G01K 1/024; G01N 27/308; G01N 33/245

(57) ABSTRACT

An apparatus including at least one vanadium oxide-doped laser-induced graphene sensor is disclosed. The sensor is configured to simultaneously monitor and decouple NO_{X} gas emission and soil temperature. The apparatus may be an electronic device including a plurality of electrical components, such as a controller unit that is connected to non-transitory memory or other type of non-transitory computer readable medium and a transceiver unit.

### CROSS-REFERENCE TO RELATED APPLICATIONS

This patent application is related to and claims the benefit of priority of U.S. Provisional Application 63/504,046, filed on May 24, 2023, the entire contents of which is incorporated by reference.

### STATEMENT REGARDING FEDERALLY SPONSORED RESEARCH DEVELOPMENT

This invention was made with government support under Grant Nos. HL154215, EB030140, DA056242 awarded by the National Institutes of Health and under Grant No. ECCS1933072 awarded by the National Science Foundation. The Government has certain rights in the invention.

### FIELD

The present disclosure generally relates to an apparatus including a vanadium oxide-doped laser-induced graphene sensor configured to decouple detection of nitrogen loss and temperature, and methods of making and using thereof.

### BACKGROUND

One key issue in traditional agricultural practices is the large amount of nitrogen fertilizers applied to soil for promoted food production, which result in excessive nitrogen oxides (NO_{X} including NO and NO₂) emissions and cause environmental pollution (e.g., photochemical smog and acid rain). Another key issue in agriculture is monitoring soil temperature, as soil temperature influences physical, chemical, and microbiological processes in soil, which are key to the growth of plants and crops.

### SUMMARY

We have determined that there is a demanding need to develop a multi-parameter sensor to monitor NO_{X} gas emission and soil temperature for regions of soil having plants that are being grown for efficient fertilization in smart and precision agriculture.

While recent advances in gas sensors have explored various types of nanomaterials, including transition metal dichalcogenides (TMDs), metal-organic frameworks (MOFs), metal oxides, black phosphorus, as well as transition metal carbides and carbonitrides (MXene), these nanomaterials are prepared by relatively complex and costly methods, and also need to be integrated on high-resolution interdigitated electrodes that are fabricated with photolithography in chemiresistor gas sensors. Moreover, heaters are often separately integrated into the sensors to elevate temperature for expedited gas adsorption/desorption toward real-time detection. As an alternative, a gas sensing platform based on three-dimensional porous laser-induced graphene (LIG) prepared by low-cost laser direct writing can eliminate the need for separate heaters due to large specific surface area and self-heating properties. However, it can be challenging to control the porous structure of the LIG foam when using common carbon-containing materials during laser processes.

Additionally, vanadium oxide (VO_{X}) as a transition metal oxide exhibits excellent properties, including n-type conductivity good chemical and thermal stability, and excellent thermoelectric properties. VO_{X} and its composites also show excellent performance to physically adsorb and chemically interact with several gases, including nitrogen dioxide, ammonia, hydrogen, and methane, among others. However, the application of VO_{X} and its composites is limited by complex synthesis methods.

Accordingly, we have developed an apparatus configured to simultaneously monitor and decouple NO_{X} gas emission and soil temperature based on a sensing platform including a vanadium oxide-doped laser-induced graphene sensor. A sensor can have an LIG/VO_{X} interface that can provide the sensor with a significantly enhanced response to NO_{X} and ultralow limit of detection. The sensor can also be configured to exhibit a wide detection range, fast response/recovery, good selectivity, and stability. Meanwhile, the sensor can also accurately detect temperature over a wide linear range and low limit of detection. Ultimately, we believe embodiments of the apparatus described herein can enable exciting applications in soil monitoring.

In an exemplary embodiment, an apparatus comprises a first sensor comprising a first base layer, a first film coated on the first base layer, wherein the first film comprises a first block copolymer carbon containing material and a first VO_{X} precursor compound, and at least one laser-induced graphene electrode scribed on the first film, wherein the at least one electrode is doped with VO_{X} particles. The apparatus further comprises a second sensor comprising a second base layer, a second film coated on the second base layer, wherein the second film comprises a second block copolymer carbon containing material and a second VO_{X} precursor compound, at least one laser-induced graphene electrode scribed on the second film, wherein the at least one electrode is doped with VO_{X} particles, and a membrane configured to encapsulate the second sensor.

In some embodiments, the first sensor is configured to collect nitrogen oxide concentration data.

In some embodiments, the second sensor is configured to collect temperature data.

In some embodiments, the first sensor is configured to self-heat.

In some embodiments, the membrane is configured to block a permeation of nitrogen oxide gas molecules.

In some embodiments, the membrane has a thickness between 5-20 μm.

In some embodiments, the first sensor and the second sensor are integral with each other such that the first base layer is integral with the second base layer and the second film is integral with the first film.

In some embodiments, the first block copolymer carbon containing material and the second block copolymer carbon containing material comprise F-127-resols.

In some embodiments, the first film and the second film have thicknesses between 20-50 μm.

In some embodiments, the first base layer and second base layer are a silicon material.

In some embodiments, the first sensor has a limit of detection of 3 ppm NO₂ at room temperature.

In some embodiments, the first sensor has a detection range of 3 ppb to 5 ppm NO₂.

In some embodiments, the first sensor has a response/recovery time of 217/650 s to 1 ppm NO₂ at room temperature.

In some embodiments, the second sensor has a detection limit of 0.2° C.

In some embodiments, the second sensor has a detection range from 10-110° C.

In an exemplary embodiment, an electronic sensing device comprises a controller unit; at least one sensor connected to the controller unit, the at least one sensor comprising a base layer, a film coated on the base layer, wherein the film comprises a block copolymer carbon containing material and a VO_{X} precursor compound, and laser-induced graphene foam scribed on the film, wherein the foam is doped with VO_{X} particles; a non-transitory computer readable medium connected to the controller unit; a transceiver unit connected to the controller unit; and a power source.

In some embodiments, the transceiver unit comprises a Bluetooth transceiver unit or a radio frequency identification transceiver unit.

In some embodiments, the power source is a solar powered battery.

In some embodiments, the at least one sensor comprises a first sensor and a second sensor, wherein the first sensor is configured to collect nitrogen oxide concentration data and the second sensor is configured to collect temperature data.

In some embodiments, the electronic device is configured to transmit collected nitrogen oxide concentration data and collected temperature data to an input/output device.

In an exemplary embodiment, a method of collecting soil data comprises providing the electronic sensing device; collecting nitrogen oxide concentration data via the first sensor and collecting temperature data via the second sensor; and transmitting the collected nitrogen oxide concentration data and the temperature data to an input/output device or a central computer device for evaluation of the collected nitrogen oxide concentration data and the collected temperature data.

In some embodiments, the step of transmitting the collected nitrogen oxide concentration data and the temperature data to the input/output device or the central computer device comprises establishing a wireless communication between the electronic device and the input/output device by positioning the input/output device in proximity to the electronic device; and transmitting the collected nitrogen oxide concentration data and the temperature data via the transceiver unit, wherein the transceiver unit a Bluetooth transceiver unit.

In some embodiments, the establishing of the wireless communication between the electronic device and the input/output device or the central computer device comprises positioning the input/output device in proximity to the electronic device via a drone or vehicle.

In some embodiments, the transmitting the collected nitrogen oxide concentration data and the temperature data to the input/output device or the central computer device comprises establishing a wireless communication between the electronic device and the input/output device by positioning the input/output device in proximity to the electronic device; and transmitting the collected nitrogen oxide concentration data and the temperature data via the transceiver unit, wherein the transceiver unit a radio frequency identification transceiver unit.

In some embodiments, the establishing a wireless communication between the electronic device and the input/output device or the central computer device comprises positioning the input/output device in proximity to the electronic device via a drone or vehicle.

In an exemplary embodiment, an apparatus comprises a first sensor device comprising a first base layer, a first film coated on the first base layer, wherein the first film comprises a first block copolymer carbon containing material and a first VO_{X} precursor compound, and a first set of laser-induced graphene electrodes positioned in the first base layer and/or first film to detect temperature data; a second set of laser-induced graphene electrodes positioned in the first base layer and/or first film to detect nitrous oxide concentration data; a membrane positioned to encapsulate the first set of electrodes.

In some embodiments, the first sensor is configured to self-heat.

In some embodiments, the membrane is configured to block a permeation of nitrous oxide gas molecules.

In some embodiments, the first block copolymer carbon containing material and the second block copolymer carbon containing material comprise F-127-resols.

In some embodiments, the first set of electrodes and second set of electrodes are doped with VO_{X} particles.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 is a perspective view of an exemplary embodiment of a sensor.

FIG. 2 is a perspective view of an exemplary embodiment of a sensor.

FIG. 3 is a perspective view of an exemplary apparatus including a plurality of sensors.

FIG. 4 is a schematic illustration showing an exemplary method of making an exemplary embodiment of a sensor.

FIG. 5A is a schematic block diagram illustrating an exemplary embodiment of a system for transmitting sensor data collected by a sensor.

FIG. 5B is a schematic block diagram illustrating an exemplary embodiment of a system for transmitting sensor data collected by more than one sensor.

FIG. 5C is a schematic block diagram of an exemplary embodiment of an apparatus utilizing a plurality of electronic devices 200 that each have a sensor 100 for providing data to at least one input/output device 210 for collection and evaluation of the sensor data for monitoring soil conditions of soil (SOIL) in which plants (e.g. at least one crop) is being grown. The collected sensor data can be evaluated by the input/output device 210 or a central computer device 212 that may be communicatively connected to the input/output device 210.

FIG. 6 is a schematic block diagram of exemplary electrical components an exemplary sensor.

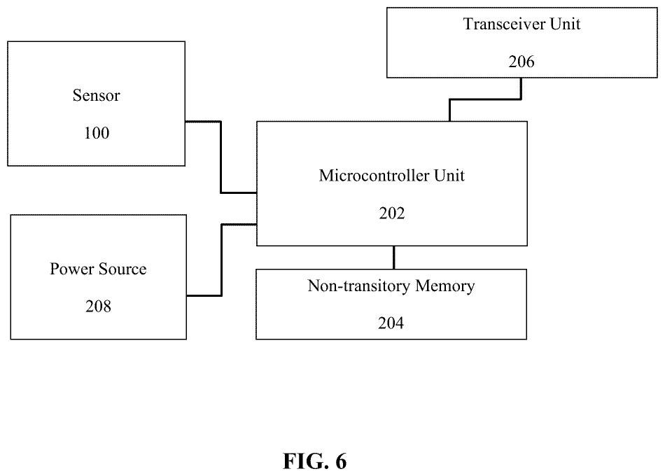

FIG. 7 is a schematic diagram showing fabrication and structure of an exemplary sensor.

FIG. 8 is a graph illustrating film thickness as a function of spin coating speed (error bars from three samples).

FIG. 9 is a schematic diagram showing the decoupling of temperature and gas of an exemplary sensor.

FIG. 10 is a schematic diagram showing soil NO_{X} gas and temperature detection of an exemplary sensor.

FIG. 11 shows scanning electron microscope (SEM) images of LIG and VOX-doped LIG.

FIG. 12 is a graph illustrating X-ray photoelectron spectroscopy (XPS) spectra of V-2p for VO_{X}-doped LIG with a zoom-in shown.

FIG. 13 is a graph illustrating XPS of V 2p regions for VO_{X}-doped LIG.

FIG. 14 is a graph illustrating Raman spectrum of VOX-doped LIG with a zoom-in shown in the inset.

FIG. 15 is a graph illustrating XRD patterns of VO_{X}-doped LIG.

FIG. 16 is a graph illustrating energy-dispersive spectroscopy (EDS) spectrum of VO_{X}-doped LIG for C, O, S, and V.

FIG. 17 is a graph illustrating response curves of the gas sensor based on pure LIG with different film thicknesses to 1 ppm NO₂.

FIG. 18 shows infrared images showing the spatial distribution of the temperature in the sensing region from different self-heating conditions: a 20.2° C., b 30.1° C., c 40.7° C., and d 50.3° C.

FIG. 19 is a graph illustrating a response curve showing the calculation of response and recovery times of the VO_{X}-doped LIG gas sensor to 1 ppm NO₂ at 30° C.

FIG. 20 is a graph illustrating a comparison in the response between the gas sensors based on LIG and VOX-doped LIG to 1 ppm NO₂.

FIG. 21 is a graph illustrating a response curve of the gas sensor to determine the response/recovery time.

FIG. 22 is a graph illustrating a dynamic response test in the presence of NO₂ from 1 to 5 ppm at room temperature.

FIG. 23 is a graph illustrating a response curve of VO_{X}-doped LIG sensor under different operating humidity at room temperature.

FIG. 24 is a graph illustrating a repeatability test to 0.5 ppm NO₂ for five consecutive cycles.

FIG. 25 is a graph illustrating a dynamic response test to NO₂ from 300 to 700 ppb at room temperature.

FIG. 26 is a graph illustrating the linear fit of FIG. 25 (error bars from three samples).

FIG. 27 is a graph illustrating response curves of the VO_{X}-doped LIG sensor to 1 ppm NO₂ under different operating relative humidity at 50° C.

FIG. 28 is a graph illustrating an experimental demonstration of the ultralow limit of detection to 3 ppb NO₂ at room temperature.

FIG. 29 is a graph illustrating a selectivity test to NO_{X} over a wide range of other gaseous molecules.

FIG. 30 is a graph illustrating current-voltage (I-V) curves of pure LIG and VO_{X}-doped LIG sensors.

FIG. 31 is a graph illustrating real-time response curves of the VO_{X}-doped LIG gas sensor to 1 ppm NO₂ at different operating temperatures.

FIG. 32 is a graph illustrating linear fit of the resistance change in the VO_{X}-doped LIG sensor with the temperature from 30 to 50° C. (error bars from three samples).

FIG. 33 is a graph illustrating response/recovery properties of the VO_{X}-doped LIG gas sensor to 1 ppm NO₂ at different operating temperatures.

FIG. 34 is a graph illustrating response of the VO_{X}-doped LIG sensor with and without the encapsulation membrane to RH 80%.

FIG. 35 is a graph illustrating a comparison in the VO_{X}-doped LIG sensor response to 30, 40, and 50° C. between the sensors with different film thickness encapsulation layer.

FIG. 36 is a graph illustrating response of the VO_{X}-doped LIG gas sensor to 1 ppm NO₂ in different humidity levels at 22 and 50° C. (error bars from three samples).

FIG. 37 is a diagram showing a temperature region of the VO_{X}-doped LIG sensor in a decoupling experiment.

FIG. 38 is a graph illustrating long-term stability test of the gas sensor to 1 ppm NO₂ for 16 days (measurement of 2000 s in each day).

FIG. 39 is a schematic showing band diagrams of the VO_{X}-doped LIG before contact.

FIG. 40 is a schematic showing band diagrams of the VO_{X}-doped LIG after contact.

FIG. 41 is a graph illustrating the difference in the sensor response to different fertilizer levels at room temperature.

FIG. 42 is a graph illustrating the adsorption energy of different gas molecules adsorbed on VO_{X}-doped LIG.

FIG. 43 is a graph illustrating the normalized relative resistance change (ΔR/R0, %) of the sensor at different temperatures over multiple cycles.

FIG. 44 is a graph illustrating the calibration curve of the sensor to temperature from 30 to 110° C.

FIG. 45 is a graph illustrating the linear fit of FIG. 44 to determine the sensitivity (error bars from three samples) with the inset to show the resistance change during continuous heating.

FIG. 46 is a graph demonstrating a sensor's ability to detect a small differential temperature change ΔT of 0.2° C.

FIG. 47 is a graph illustrating a repeatability test of the sensor over five heating-cooling cycles between 40 and 70° C.

FIG. 48 is a graph illustrating a repeatability test of the sensor in various heating-cooling cycles.

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

Embodiments generally relate to an apparatus including a vanadium oxide (VO_{X})-doped laser-induced graphene sensor. The apparatus is configured to monitor both nitrogen utilization and temperature. For example, in agricultural applications, the apparatus is configured to effectively and accurately decouple soil nitrogen loss and soil temperature when the two stimuli are simultaneously present in large-scale soil monitoring such that the apparatus may provide for crop health monitoring and essential timely intervention.

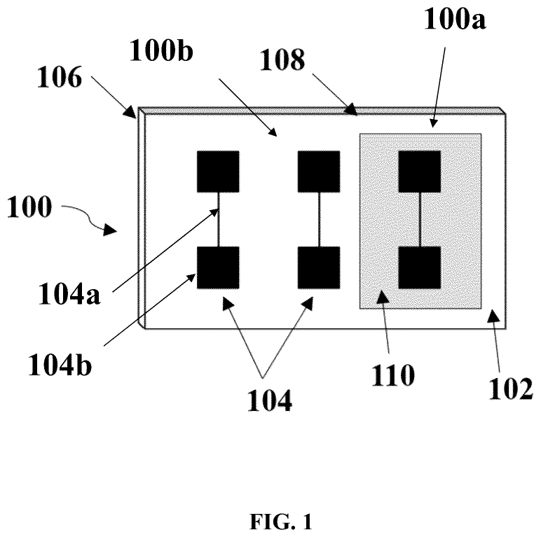

Referring to FIGS. 1-3, embodiments of the apparatus include at least one sensor 100. The sensor 100 is a laser-induced graphene (LIG) sensor provided by laser-scribing a substrate 102 using a laser (e.g., CO₂ laser), thereby forming at least one LIG electrode 104. It is contemplated that the LIG electrode 104 may be LIG foam and have a three-dimensional porous structure. The laser may be used to scribe any programmable shape and/or configuration into the substrate 102.

The substrate 102 includes a base layer 106 and a film 108 coated on the base layer 106. The base layer 106 can be a temporary layer or a permanent layer. For example, the sensor 100 may be fabricated on a temporary substrate and subsequently peeled off and/or transferred to a permanent substrate. It is contemplated that the permanent substrate is flexible. The base layer 106 may be a temporary layer such as a silicon material (e.g., SiO₂), glass, or any other suitable material. The base layer 106 may alternatively be a permanent substrate such as a silicone rubber (e.g., silicone elastomers), hydrogels, or any other suitable material, such as a polymeric material.

The film 108 may have a thickness of 20-50 μm, and preferably 35 μm. The film 108 may include a carbon containing material, such as Pluronic F-127, polyimide (PI), polyamide-imide (PAI), polyethersulfone (PES), polyphenylene sulfide (PPS), or any other suitable material. In preferred embodiments, the carbon containing material is a block copolymer (e.g., rather than a single monomer), such as Pluronic F-127. For example, the carbon containing material may be a Pluronic F-127-resols material, which allows for a tunable mass ratio of copolymer to resols mixture and control of resulting three-dimensional structure and pore size distribution of the LIG electrode 103.

The film 108 further includes a precursor compound, such as a VO_{X} precursor compound. In preferred embodiment, the precursor compound is V₅S₈. It is contemplated that laser-scribing the film 108 including the carbon containing material and the VO_{X} precursor compound results in a VO_{X}-doped LIG electrode 104. For example, laser-scribing the film 108 results in the precursor compound (e.g., V₅S₈) being oxidized to VO_{X} and in a uniform doping and anchoring of VO_{X} particles within the LIG electrode 104.

The at least one LIG electrode 104 may be configured to collect gas data, such as nitrogen oxide (NO_{X}) concentration. It is contemplated that NO_{X} emissions are the result of natural processes occurring in soil, and due to concerns about the crop yield and overall environmental impacts of NO_{X}, its management is becoming increasingly important. The sensor 100 may be configured to easily adsorb NO_{X} gas molecules onto the composite structure of the VO_{X}-doped LIG foam 102 and therefore detect soil nitrogen loss.

The at least one LIG electrode 104, may be configured to collect temperature data, such as soil temperature. The sensor 100 may be configured to detect and measure resistance of an LIG electrode, which may change with temperature and depends on the temperature coefficient of resistance of the LIG. It is contemplated that there may be a linear or substantially linear relationship between temperature and resistance in a given range of interest.

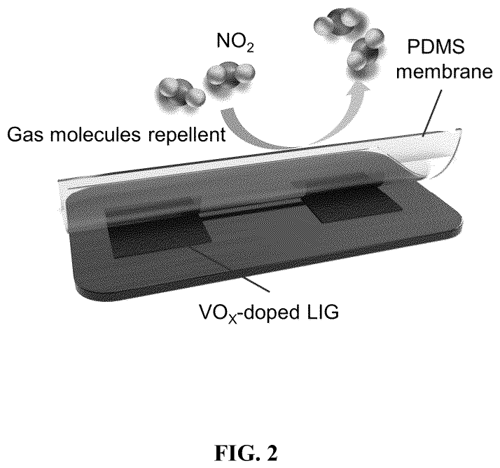

In exemplary embodiments, the sensor 100 may be encapsulated by a membrane 110 (see FIG. 2). In some embodiments, at least the LIG electrode(s) 104 may be encapsulated by a membrane 110. It is contemplated that encapsulated means enclosed and/or not exposed to surroundings. For example, the membrane 110 may act as a closed face over the otherwise open face of the sensor 100. The membrane 110 is configured to block the permeation of gas molecules such that the sensor 100 may respond only to variations in temperature (e.g., without being affected by NO_{X}). Accordingly, it is contemplated that a sensor 100 encapsulated by the membrane 110 is advantageously configured to collect temperature data. The membrane 110 may be formed from a flexible polymer, such as polydimethylsiloxane (PDMS) or any other suitable polymer. The membrane 110 may be attached to the sensor using an adhesive, such as a PDMS adhesive, a glue, or any other suitable adhesives. The membrane 110 may have a thickness of 1-20 μm, and preferably 10 μm. It is contemplated that rapid heat transport in the membrane 110 has minimal effect on temperature sensing.

In some embodiments, the sensor 100 and/or the at least one LIG electrode 104 may not be encapsulated such that the sensor 100 is exposed to NO_{X} gas molecules. Accordingly, it is contemplated that a sensor 100 not encapsulated by a membrane 110 is advantageously configured to collect gas data, such as NO_{X} concentration.

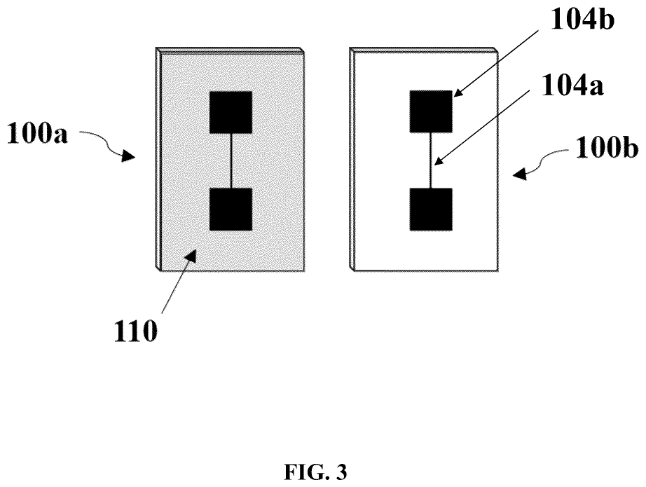

In some embodiments, the sensor 100 may be self-heated. such that the sensor 100 operates at elevated temperatures. In particular, it is contemplated that when a current or voltage is applied on the LIG electrode 104, a Joule heating effect—in which electric energy is converted into thermal energy as the current or voltage is applied—may occur to measure the electrode's resistance change. In some embodiments, the sensor 100 may be self-heated, preferably in a central sensing region 104a, by designing the resistance of the central sensing region 104a much larger than an outside connection region 104b (see FIGS. 1 and 3). This can be achieved by, for example, choosing smaller thickness/width in the central sensing region 104a and/or coating the outside connection region 104b with highly conductive materials, such as metals. Operating at elevated temperatures via self-heating (e.g. heating that occurs via ambient conditions) can be advantageous as water molecules associated with relative humidity are in competition with NO_{X} gas molecules for adsorption onto the composite structure of the VO_{X}-doped LIG electrode 104. Operating at elevated temperature may therefore create thermal radiation to drive the water molecules away from the sensor 100. Accordingly, it is contemplated that a sensor 100 that is self-heating/operates at an elevated temperature is advantageously configured to collect/detect data related to nitrogen loss.

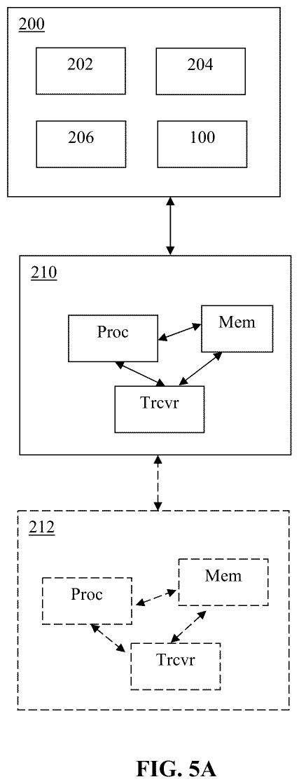

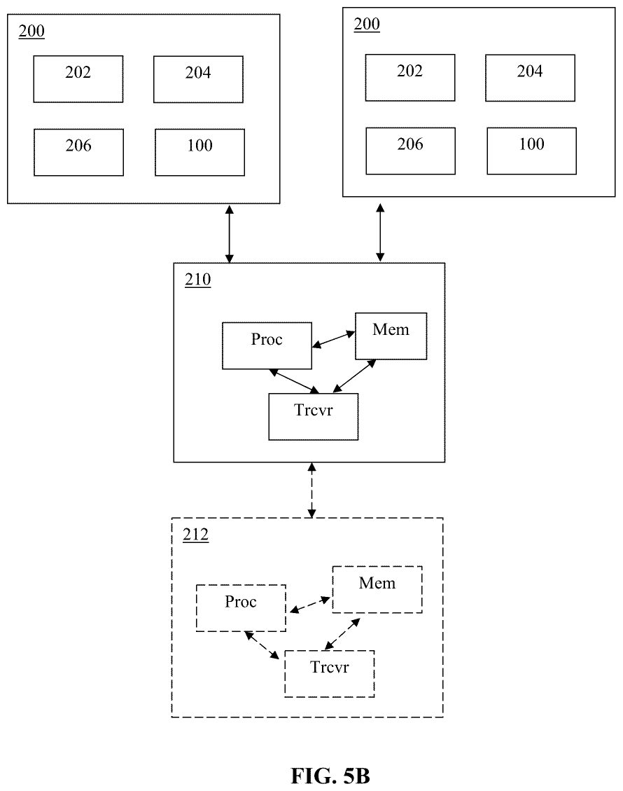

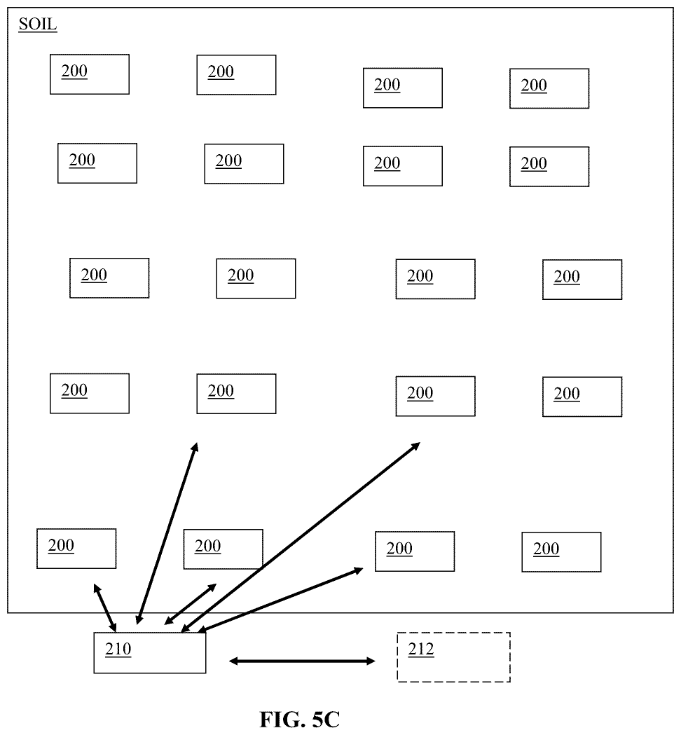

Embodiments of the apparatus can be configured to include at least one sensor 100, such as a plurality of sensors 100 and/or an array of sensors 100 (see FIG. 5C). For example, in embodiments including a plurality of sensors 100, each sensor 100 may operate and collect data independently with no interference between their input signals.

In one embodiment, the sensor 100 can include at least a first sensor 100a and a second sensor 100b. These sensors 100 can be integral with each other such that they are defined on the same substrate 102 (a first base layer is integral with a second base layer, and a first film is integral with a second film) as shown in FIG. 1, for example. The first sensor 100a can include a set of LIG electrodes 104, which can be connected to each other and configured to facilitate collection of data related to temperature. The first set of LIG electrodes 104 can be positioned on or in a base layer and/or a film formed on the base layer. The second sensor 100b can include a set of LIG electrodes 104, which can be connected to each other and configured to facilitate collection of data related to nitrous oxide concentration or other type of gas data. The second set of LIG electrodes 104 can be positioned on or in a base layer and/or a film formed on the base layer. The first set of electrodes can be encapsulated by a membrane 110 and the second set of electrodes may not be encapsulated by the membrane 110.

For example, the first sensor 100a may be configured to collect temperature data, and the second sensor 100b may be configured to collect gas data. In this embodiment, the first sensor 100a may be encapsulated by membrane 110, as described above, and the second sensor 100b may not be encapsulated. For example, the membrane 110 can be positioned on the body of the sensor to encapsulate only the first sensor 100a for detection of temperature data without having moisture or other elements interfering with the sensor's ability to detect the temperature data while the second sensor 100b is not encapsulated so that it can collect NOx related data measurements. Embodiments can have such a configuration so that the detection of temperature and NOx data can be decoupled from each other while the sensor 100 is also able to simultaneously monitor both nitrogen utilization in the soil and temperature of the soil.

In application, the apparatus may be positioned at or near a soil surface to accurately monitor soil temperature and NO_{X} emission. In exemplary embodiments, a plurality of apparatuses can be used to collect data regarding various locations of an area of land (e.g., field). For example, a first sensor 100 may be positioned at a first location, a second sensor 100 may be positioned at a second location, etc., such that the soil temperature and NO_{X} emission of an entire area of land may be monitored. In some embodiments, tens, hundreds, or thousands of the sensors 100 can be positioned in the soil along various different spaced apart locations to monitor the NOx and temperature conditions at those locations to collect data related to plant fertilization and/or plant growth for the plants growing in those different spaced apart locations.

Regarding collection of gas data, the sensor 100 as described herein can have a low limit of detection of NO_{X} gas concentration, such as 3 ppm NO₂ at room temperature. It is contemplated that the VO_{X}/LIG significantly enhances the performance of the sensor 100, allowing for the detection of the ultra-low concentration NO₂. The sensor 100 can be configured to have a wide detection range, e.g. from 3 ppb to 5 ppm NO₂ or other suitable range. The sensor 100 can also have a fast response/recovery time, such as, e.g. 217/650 s to 1 ppm NO₂ at room temperature.

Embodiments of the sensor 100 can be configured to provide a good selectivity for NO_{X} molecules, exhibiting a ten-fold response to NO₂ over other interfering gases. It is contemplated that the sensor 100 can also provide excellent selectivity for NO_{X} due to the lowest unoccupied molecular orbital of NO_{X} being lower than that of other gases resulting in more electrons transferred from the sensor 100.

Regarding collection of temperature data, embodiments of the sensor 100 can have a low detection limit (e.g. of 0.2° C., etc.) and a wide detection range, e.g. from 10° C.-110° C., etc.

Referring to FIGS. 5A-5C, the apparatus can include at least one electronic device 200. Each electronic device 200 can include a sensor 100 incorporated therein. The electronic device 200 may include a plurality of electrical components, such as a controller unit 202 that is connected to non-transitory memory 204 or other type of non-transitory computer readable medium and a transceiver unit 206. The transceiver unit 206 may be a Bluetooth transceiver unit or a radio frequency identification (RFID) transceiver unit. The controller unit 202 interfaces with the rest of the components of the electronic device 200. The electronic device 200 may further include a power source 208 for powering the controller unit 202 and electric components thereof. The controller unit 202 can receive data from the sensor 100 and store that data in its memory. The electronic device 200 can be configured to transmit the collected data to another device or evaluate the collected data to determine temperature of the soil and NOx content of the soil based on the collected sensor data.

The power source 208 of the electronic device can be a battery, such as a rechargeable battery, configured to provide a source of voltage to power the electronic device 200 and the components of the electronic device 200. In some embodiments, the power source 208 may be a solar powered battery. It is contemplated that the power source 208 may be positioned within the electronic device 200 and/or on an external surface of the electronic device 200. For example, if the power source 208 is a solar powered battery, the power source 208 may advantageously be positioned on an external surface of the electronic device 200.

Each sensor 100 or plurality of sensors 100 of an electronic device 200 can be communicatively connected to the controller unit 202 and/or the non-transitory computer readable medium 204 such that the sensors 100 interface with and provide data to the controller unit 202 and/or the non-transitory computer readable medium 204 (e.g., via a serial peripheral interface (SPI), an inter-integrated circuit (I2C), a universal asynchronous receiver-transmitter (UART), etc.). The controller unit 202 can be a processor or other type of control hardware element.

In some embodiments, the controller unit 202 can wirelessly transmit the data collected by the sensor 100 or plurality of sensors 100 to an input/output device 210 (e.g. a smart phone, tablet, laptop computer, personal computer, computer device of a drone or a vehicle that may move through a region of soil having one or more of the electronic devices 200, etc.) using the transceiver unit 206. While FIGS. 5A and 5B are schematic block diagrams including one sensor 100 and two sensors 100, respectively, the disclosure encompass all embodiments with at least one sensor 100.

It is contemplated that the data collected by the sensor 100 or the plurality of sensors 100 may be processed by a low pass filter to remove any noise before being wirelessly transmitted to the input/output device 210.

The transceiver unit 206 is configured as a wireless interface for the transmission of sensor data via the electronic device's 200 wireless connection with an input/output device 210. In some embodiments, a user may aid in establishing a wireless communication between the electronic device 200 and the input/output device 210 by positioning the input/output device 210 in sufficient proximity to the electronic device 200. In alternative embodiments, a moveable device (e.g., drone, such as a flying drone or a drone that is moveable on the ground, vehicle, etc.) may aid in establishing a wireless communication between the electronic device 200 and the input/output device 210 by positioning the input/output device 210 in sufficient proximity to the electronic device 200.

In some embodiments, the data collected by the sensor 100 or plurality of sensors 100 may be continuously streamed to the input/output device 210. It is contemplated that the electronic device may therefore provide real-time data collection. In other embodiments, the data collected by the sensor 100 or plurality of sensors 100 may be periodically streamed to the input/output device 210 (e.g., non-continuously at pre-determined intervals, every five seconds, every minute, etc.). The data collected by the sensor 100 or plurality of sensors 100 can also be stored within the non-transitory computer readable medium of the electronic device 210 for an extended period of time before being transmitted to the input/output device 210.

It should be appreciated that the input/output device 210 is a computer device that has at least one processor (Proc) connected to at least one non-transitory computer readable medium (e.g. memory, a hard drive, etc.) (Mem) and at least one transceiver unit (e.g. a network transceiver, etc.) (Trcvr).

It is contemplated that the data may alternatively or subsequently be sent to a central computer device 212 (e.g. server, an operator work station, etc.) that can be connected to the input/output device 210 (e.g. via a network connection and application programming interface (API) the central computer device may have with the input/output device 210 running the application, etc.). The central computer device 212 may be configured to store the data of each electronic device 200 and/or sensor 100 it receives. The forwarding of such data can be provided via a communication connection the electronic device 200 directly has with an input/output device 210 such that data is sent to the central computer device 212 via the input/output device 210.

It should be appreciated that the central computer device 212 can be configured as a computer device that has at least one processor (Proc) connected to at least one non-transitory computer readable medium (e.g. memory, a hard drive, etc.) (Mem) and at least one transceiver unit (e.g. a network transceiver, etc.) (Trcvr). The central computer device 212 can be configured as a database server, a workstation, a desktop personal computer, a laptop computer, or other suitable computer device that can receive the sensor data and evaluate the sensor data for generation of output that can be displayed or otherwise output to an operator to provide NOx and temperature data based on the sensor data the computer device has received.

It is contemplated that once the data is transmitted to the input/output device 210 and/or the central computer device 212, the temperature and NOx data collected by the sensor(s) 100 may be analyzed and evaluated. For example, the data can be processed using artificial intelligence or machine learning algorithms stored on the input/output device 210 and/or the central computer device 212. In particular, the input/output device 210 and/or central computer device 212 may run a program that uses the collected data along with a module trained via a machine learning process that received the collected data and processes that data to determine an output.

In some embodiments, the input/output device 210 and/or the central computer device 212 can be configured to generate a graphical user interface (GUI) on a display for providing information to a user regarding collected data. For example, the electronic device 200 may transmit data collected from the sensor 100 or plurality of sensors 100 the input/output device 210 and/or the central computer device 212, and the collected data may be displayed in the form of a graph, chart, text, or other display via the GUI. The GUI may further display outputs or other collected data to the user.

In yet other embodiments, it is contemplated that the sensor(s) 100 can be integrated into an electronic device 200 that can have sufficient processing power and input devices and output devices for analyzing the sensor data collected via the sensor(s) 100. In such a configuration, the electronic device can include code defined in its memory and run that code to evaluate the data in accordance with a pre-defined evaluation process for generation of output to a user. Such output can be displayed via a GUI that can be generated on a display of the electronic device 200.

In configurations of the apparatus that may utilize at least one input/output device 210 and/or a central computer device 212, the sensor data from one or more sensors 100 can be communicated to those devices via the electronic device(s) having the sensor(s) 100. The data can subsequently be collected and analyzed. In such embodiments, positional data or other data that identifies each sensor 100, electronic device 200, and/or the location of the collected data can also be provided for the collection and analysis of the data so that different regions of the soil being monitored can be identified and the collected data for different sensors 100 can be linked to those locations. The location data can include positional data, an identifier that is pre-defined as being linked to a location (e.g. via a database cross-reference identifier, etc.) or other type of identifier that can be utilized for linking each sensor's data to the sensor's location in a region of soil being monitored. The device analyzing the sensor data can use the location information to provide output about different specific regions within the soil being monitored.

Embodiments of the apparatus can be configured to facilitate monitoring of plant growth and fertilization utilization. For example, embodiments can detect low fertilizer conditions or poor plant growth conditions to direct further fertilization treatments in specific soil regions to avoid over fertilization of a particular region while also providing sufficient fertilization for supporting a desired level of plant growth. Embodiments can also provide data that can be relevant to providing a desired level of water or other nutrients to the plants in different regions so a user can provide a more directed and efficient level of nutrients (e.g. water, fertilizer, etc.) to different regions of soil being monitored based on the sensor data that is collected and evaluated.

Embodiments can also be configured so that the input/output device 210 or the central computer device 212 can provide empirical data concerning the sensor data to help an operator forecast and/or plan fertilizer needs for a coming season and/or make other projections concerning planting and growth of different crops.

### EXAMPLES

### Example 1: Exemplary Sensor Construction and Components

An exemplary embodiment of a sensor 100 was constructed. For this particular embodiment, the film and other elements of the sensor 100 was provided as noted herein.

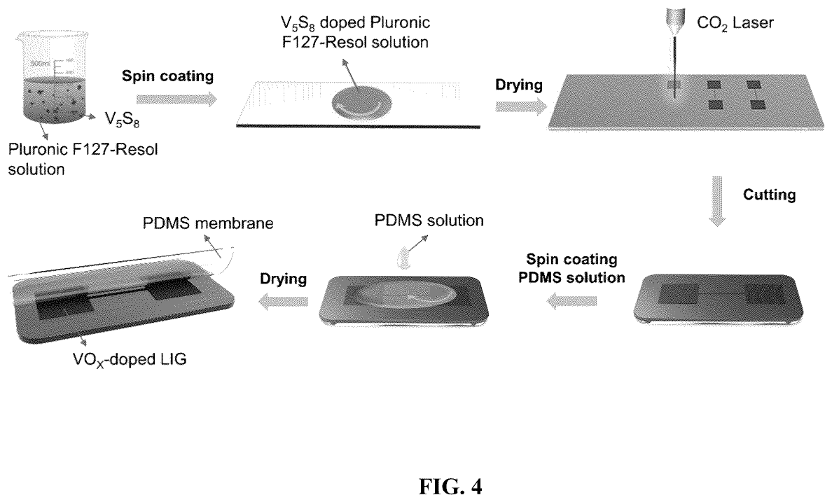

Preparation of Film: A Pluronic F127-resols solution was obtained by mixing 4 g resols and 6 g Pluronic F127 copolymer in ethanol in a water bath (40° C., 4 h) (FIG. 4). Adding V₅S₈ to the above solution followed by stirring prepared the V₅S₈-doped Pluronic F127-resols solution. V₅S₈ were purchased from Nanjing MKNANO Tech. Co., Ltd. After vacuum treatment for 20 min, spin coating the solution on a silicon (Si) substrate at varying speeds (i.e., 200, 500, 850, 1000, 1500, 2000, 2500, 3000, 3500, and 4000 rpm), followed by drying in a vacuum oven at 150° C. for 48 h, formed a uniform thin film.

Preparation of Sensor: A sensing region (width of 150 μm and length of 4.5 mm) with two square electrodes was directly created by scribing the V₅S₈-doped Pluronic F127-resols hybrid film with a CO₂ laser (Universal Laser, 10.6 μm, spot size of 127 μm, and power of 9 mW). The laser processing parameters were fixed (power of 3.0%, speed of 1%, and PPI of 500), unless specified otherwise. Copper tapes with silver paste on the square electrodes connected the sensor to the data acquisition system. The PDMS solution was prepared at room temperature with stirring using 2 g of prepolymer (a) and 0.1 g of crosslinking agent (b) in a mass ratio of 20:1. Then, the PDMS solution was spin-coated onto the sensing area of VO_{X}-LIG sensor at varying speeds (i.e., 6000, 7000, 8000, and 9000 rpm) and drying in a vacuum oven at 85° C. for 1 h. Target gas are first collected into an aluminum foil gas collecting bag and then injected into a closed chamber for static detection. Different concentrations of NO_{X} were prepared by diluting and fully mixing the commercial calibration gas of 50 ppm NO_{X} with air in the chamber (volume of 5 L). The different relative humidity values in the chamber were prepared by the saturated salt solution method. The concentration of the VOC was obtained by injecting the needed quantity of anhydrous liquid analytes into a sealed glass container using a microliter syringe. The concentration (C in ppm) of the VOC in the chamber was calculated using the following Equation (1):

$$C = \frac{2 2.4 ρ {TV}_{S}}{237 MV} , \qquad ( 1 )$$

where ρ, V_{S}, and M are the density (g ml⁻¹), volume (μL), and molecular weight (g mol⁻¹) of the anhydrous liquid VOC, T is the testing temperature (K), and V is the volume of the glass container (L) filled with the VOC.

The real-time resistance was recorded by a SourceMeter (Keithley 2400) at a constant voltage of 0.05 V. The sensor response is defined as S=ΔR/R₀ with ΔR=R−R₀, where R₀ is the initial resistance in air and R is the resistance in the target gas. The response (or recovery) time is the time taken for the sensor response to reach 90% of the response (or recovery) at saturation in the target gas (or air).

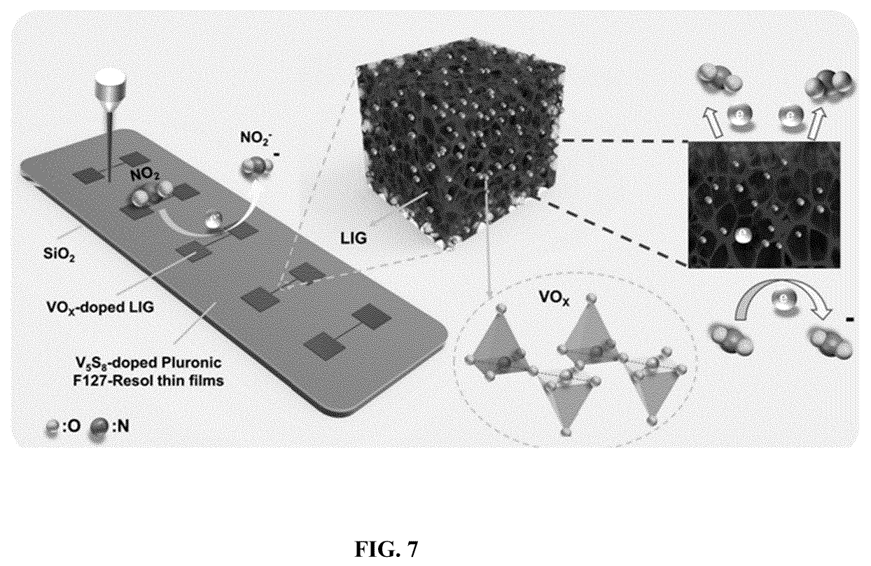

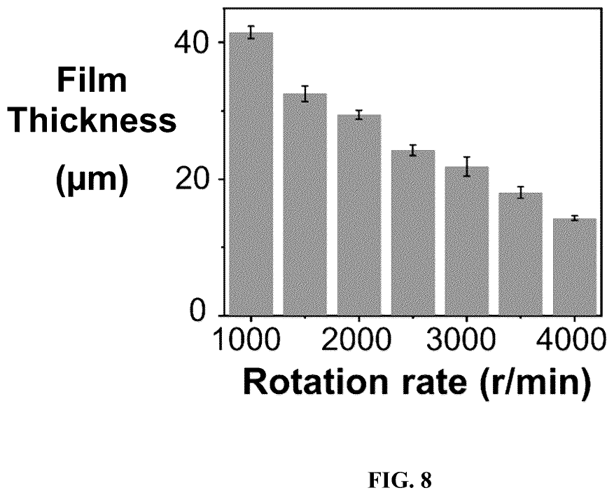

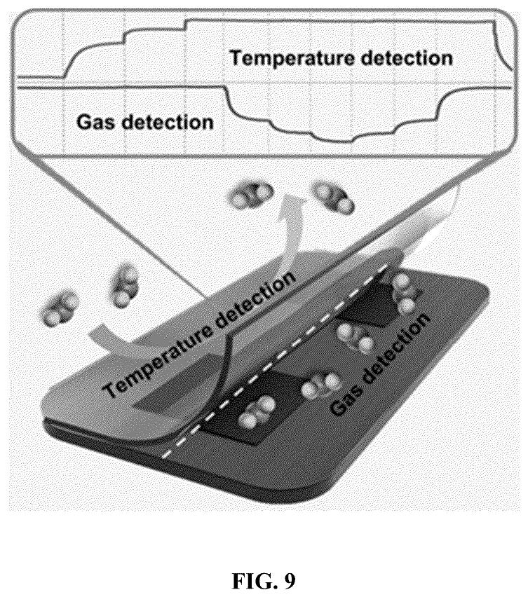

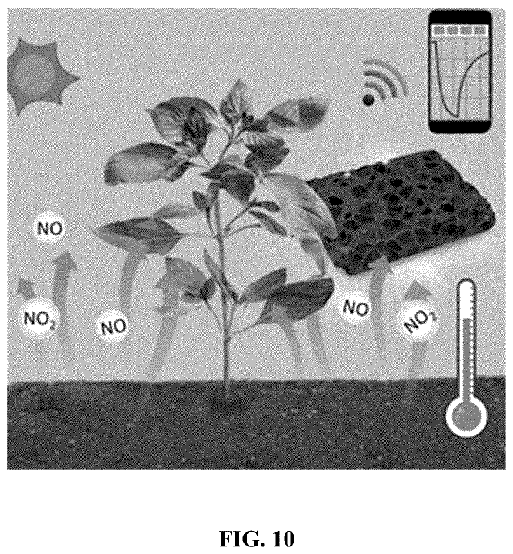

As described above, the VO_{X}-doped LIG sensor can be obtained by laser scribing the carbon-containing material such as Pluronic F127-resols doped with a VO_{X} precursor such as V₅S₈ (FIGS. 1 and 7). In brief, spin coating the V₅S₈-doped Pluronic F127-resols solution on a Si substrate at different speeds forms a thin film with different thicknesses (FIG. 8). After drying the thermosetting phenolic resin, applying the CO₂ laser in a programmed pattern on the resulting thin film creates the VO_{X}-doped LIG sensor, which can be scalable for massive production. At a scanning rate of 12.7 μm/s, V₅S₈ in the thin film is instantaneously oxidized to VO_{X}, providing uniform doping of VO_{X} in the 3D LIG foam. The sensor capable of decoupling the NO_{X} and temperature (FIG. 9) allows for NO_{X} and temperature monitoring in the soil (FIG. 10).

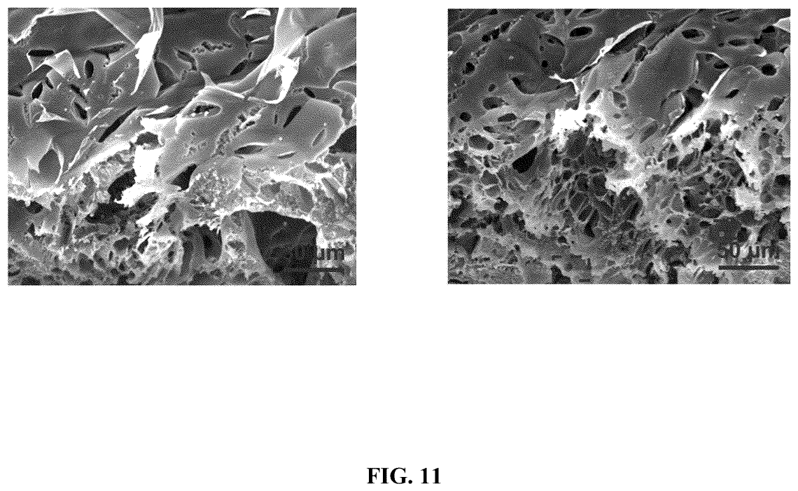

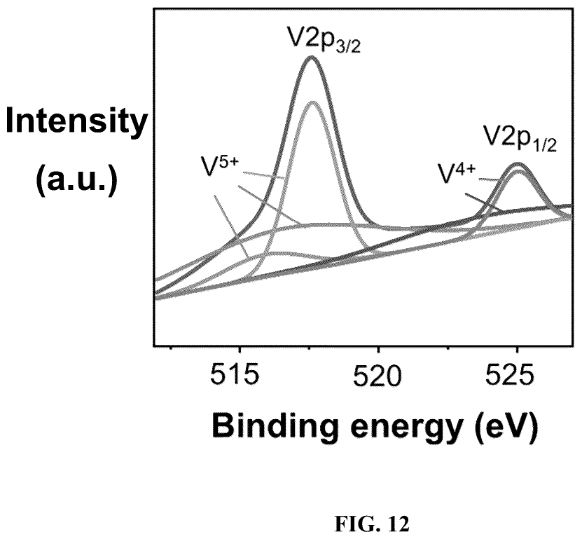

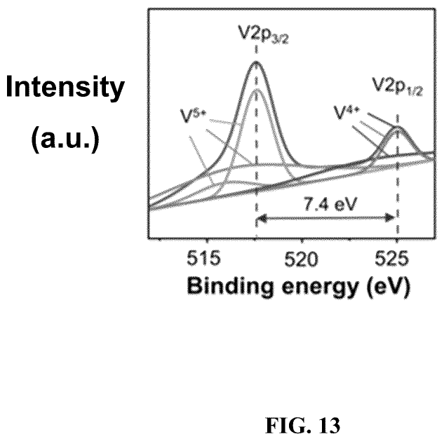

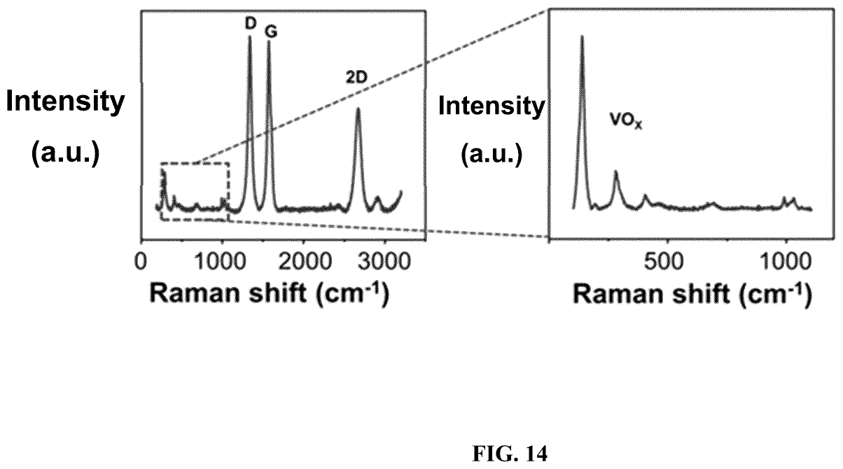

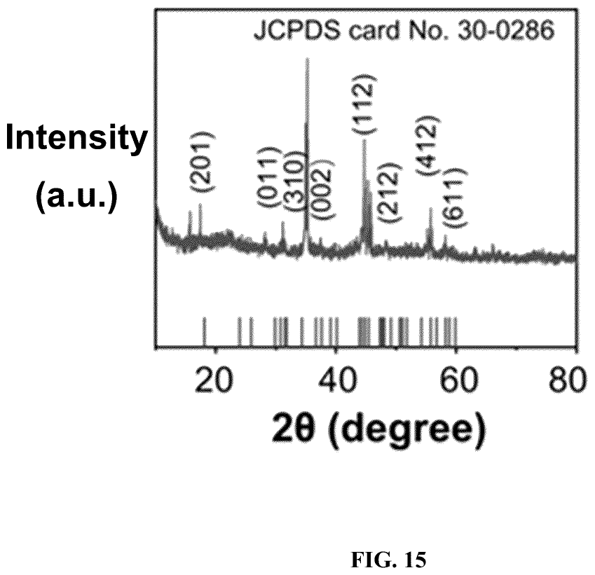

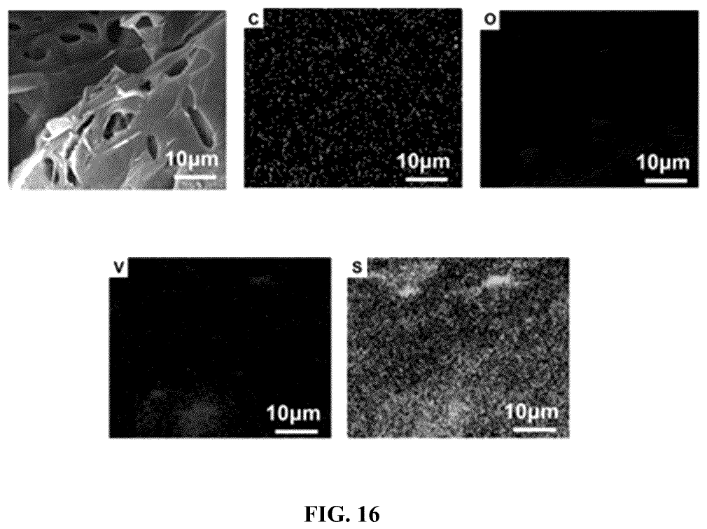

The porous-structured LIG (FIG. 11) doped with the laser-induced V5S8 and VO_{X} particles (FIG. 11, box) can be observed in the scanning electron microscope (SEM) image. Several different peaks in the C is and V 2p of XPS spectra indicate the successful formation of graphene and VO_{X} in the laser process (FIG. 12). The two V 2p peaks at the binding energy values of 517.60 eV and 525 eV in the spectrum correspond to V 2p_{3/2} and V 2p_{1/2}, respectively (FIG. 13). The observed binding energy values with a spin-orbit splitting of 7.4 eV are in good agreement with the V₅⁺ oxidation state. The spin-orbit splitting between V 2p_{3/2} and V 2p_{1/2} peak is 7.4 eV. The Raman spectra confirm the presence of few-layered graphene in VO_{X}-doped LIG (FIG. 14) by exhibiting three prominent peaks: the D (˜1338 cm⁻¹), G (˜1524 cm⁻¹), and 2D (˜2673 cm⁻¹). The enlarged Raman spectrum shows peaks at 142, 193, 281, 404, 695, and 991 cm⁻¹ to confirm the formation of VO_{X}. The combination of LIG and VO_{X} in VO_{X}-doped LIG abnormally shifts the peaks compared with the previously reported VO_{X}. The XRD patterns of VO_{X}-doped LIG show peaks at 24.0°, 30.8°, 34.3°, 36.7°, 45.4°, 47.6°, 55.8°, and 58.2° (FIG. 15), corresponding to (201), (011), (310), (002), (112), (212), (412), and (611) (JCPDS card No. 30-0286) to confirm the formation of VO_{X}. The spectra confirm the presence of VO_{X}, although it is difficult to distinguish from V₅S₈ due to the overlap in the bands. The distinct distributions of C, O, S, and V elements in the EDS spectrum in the sensing area demonstrate that V₅S₈ is partially transformed to VO_{X} during laser processing (FIG. 16).

### Example 2: Evaluating Effect of Thickness on Film on Gas Sensing Performance

We conducted a study evaluating the effect of the thickness of the film on gas sensing performance based on the exemplary embodiment of the sensor as designed above in Example 1.

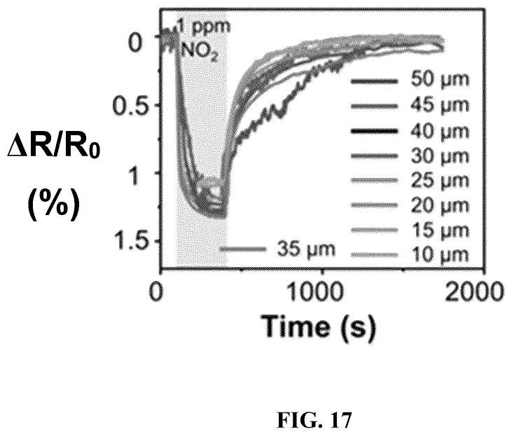

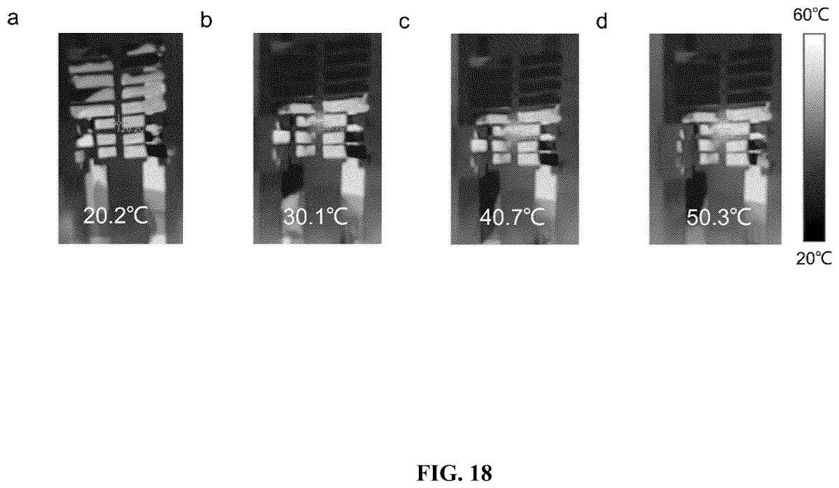

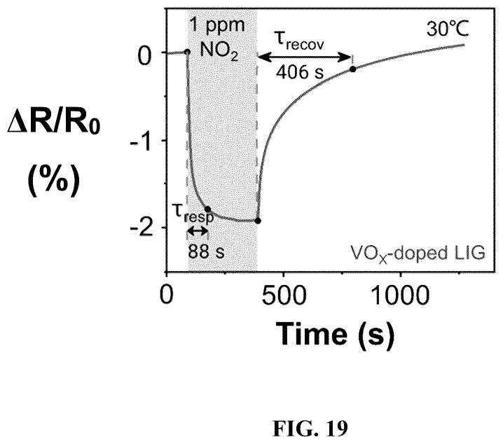

The film thickness modulates the laser-induced gas sensing area and gas permeation through the thickness, affecting the sensor performance. The LIG gas sensor with either a small or large thickness has a low response and signal-to-noise ratio (SNR) in the gas response curve to 1 ppm NO₂ at room temperature (FIGS. 17-19). On the other hand, with the increase of film thickness, the conductivity increases but gas permeability decreases to give a reduced gas response. The LIG gas sensor with a larger thickness can provide abundant gas adsorption sites and continuous gas diffusion pathways, facilitating gas analyte-induced charge carrier generation for enhanced gas-sensing properties. However, as the film thickness exceeds 35 μm, some structures in the resulting LIG become poor quality with uneven 3D microstructures, leading to unstable sensing performance. Therefore, the optimal film thickness of 35 μm is selected in the following studies.

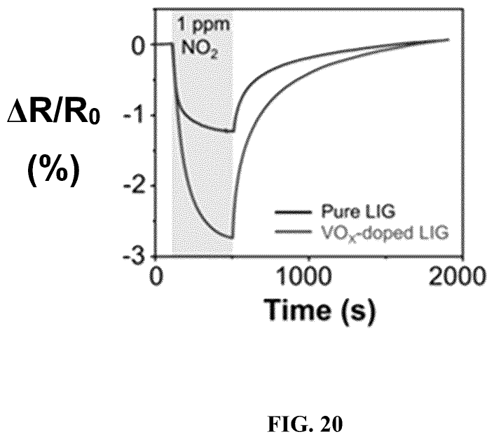

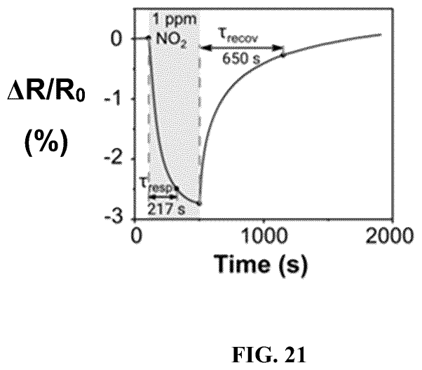

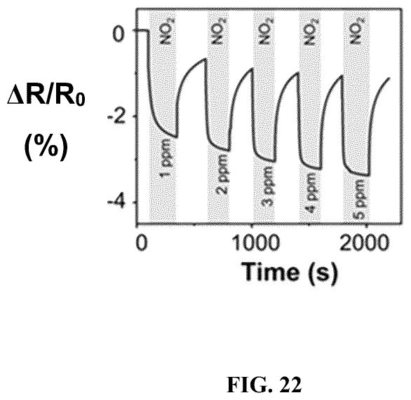

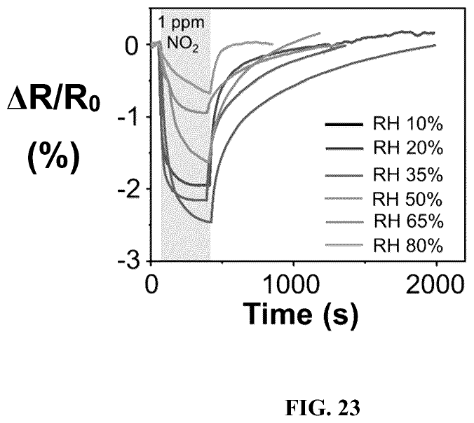

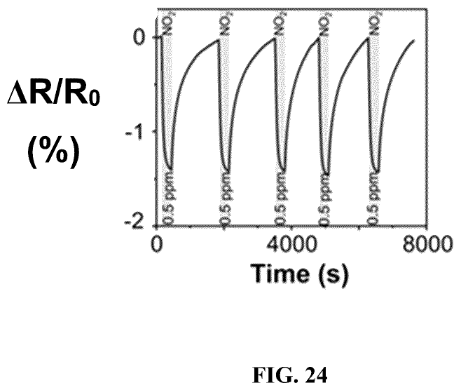

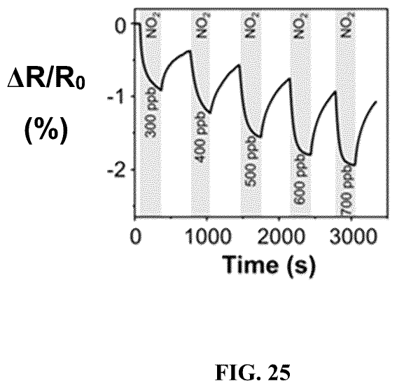

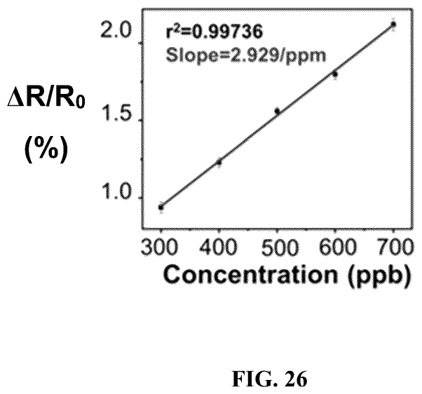

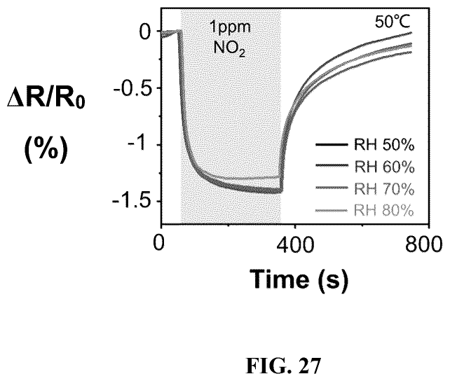

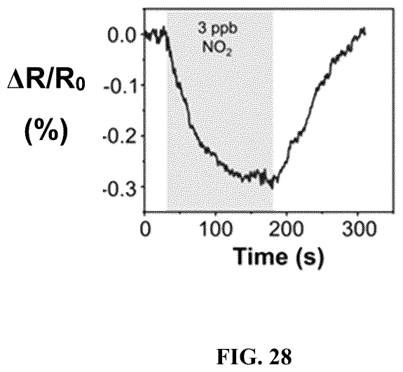

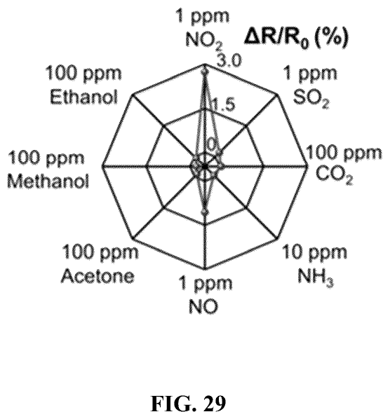

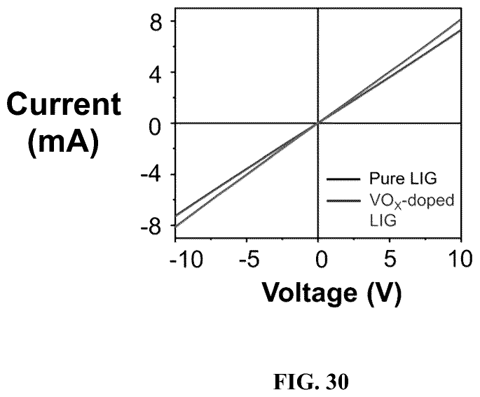

Compared with pure LIG gas sensors, the VO_{X}-doped LIG gas sensors provide a higher response, e.g., over a two-fold increase from 1.2% to 2.8% when exposed to 1 ppm NO₂ (FIG. 20). The response/recovery time of 217/650 s to 1 ppm NO₂ at room temperature is also relatively rapid (FIG. 21 As the recovery time of 300 s is sufficient to capture the characteristics of the gas sensor, this value is selected in the following studies unless specified otherwise. As the concentration is gradually increased from 1 to 5 ppm, the continuous response curve to NO₂ shows an increase from 2.5% to 5.5% (FIG. 22), indicating a good dynamic response/recovery at room temperature. The response and recovery time also increases with the increasing gas concentration from 1 to 5 ppm (FIG. 23). The incomplete recovery comes from the short time set for rapid testing. The cycling stability of the VO_{X}-doped LIG sensor is confirmed by exposure to 0.5 ppm NO₂ at room temperature (FIG. 24). The consistent response and recovery of the sensor over five cycles indicate the good steady-state response of the sensor. To provide a more accurate estimation of the theoretical limit of detection (LOD), the gas sensor is exposed to NO₂ with a progressively increased concentration from 300 to 700 ppb with a step size of 100 ppb, which gives a continuous response curve from 0.9%, 1.2%, 1.5%, 1.8%, to 2.1% (FIG. 25). The linear fit of the sensor response to the NO₂ gas concentration from 300 to 700 ppb yields a slope of 2.929 ppm⁻¹ with a correlation coefficient (R²) of 0.997 (FIG. 26). The linear fit of the gas sensor response to the lower concentration (3 ppb, 5 ppb, 7 ppb, 9 ppb, to 11 ppb) gives a slope of 8.4×10⁻⁵/ppb, which further leads to the determination of the theoretical LOD, defined as 3×RMSₙₒᵢₛₑ/slope, as 451 ppt (FIG. 27). As it is challenging to use the static gas test setup to measure the gas concentration below 1 ppb (additionally spiked gas in the ambient environment), testing of the sensor to 3 ppb NO₂ still shows a response of 0.3‰ with a signal-to-noise ratio (SNR) of 26.85 (FIG. 28). The VO_{X}-doped LIG gas sensor only gives a small response of 0.2%, 0.06%, 0.062%, 0.12%, 0.22%, and 0.09% to 1 ppm SO₂, 100 ppm CO₂, 10 ppm NH₃, 100 ppm acetone, 100 ppm methanol, and 100 ppm ethanol, respectively (FIGS. 29-30). In comparison, the significantly higher response of the sensor to NO_{X} (e.g., 2.7%/1.1% to 1 ppm NO₂/NO) highlights the excellent selectivity. The exposure of the chemiresistive VO_{X}/LIG gas sensor to an oxidizing NO_{X} gas results in the extraction of the electrons in the valence band of the LIG to the adsorbed NO_{X} and from VO_{X} to LIG. The lowest unoccupied molecular orbital (LUMO) determines the number of transferred electrons. As the LUMO of NO_{X} gas molecules is lower than that of other gases, more electrons are transferred from the LIG to give a larger response and high selectivity to NO_{X}. As a result, the VO_{X}-doped LIG gas sensor with an ultralow LOD and a high selectivity outperforms the other NO_{X} gas sensors based on different nanomaterials.

### Example 3: Evaluating Effect of Operating Temperature from Self-Heating on Gas Sensing Performance

We conducted a study evaluating the operating temperature from self-heating on gas sensing performance based on the sensor as designed above in Example 1.

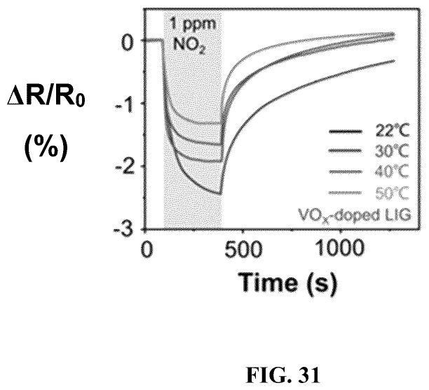

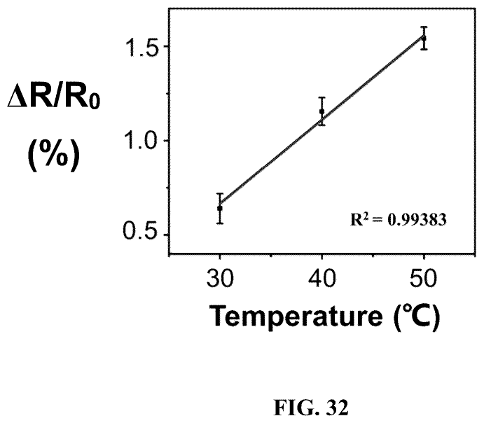

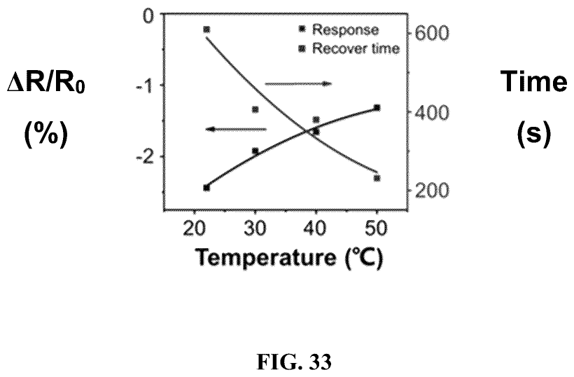

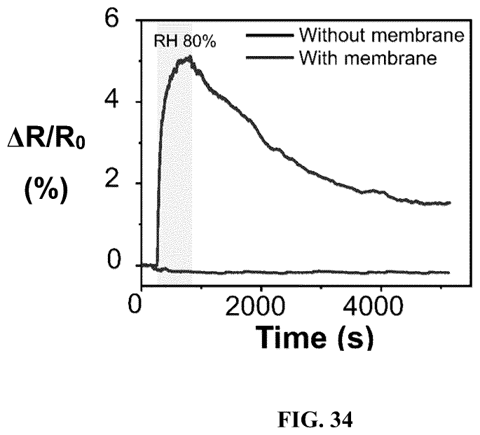

As temperature is often used to modulate the gas sensing performance, self-heating of the LIG was explored to modulate the temperature by changing the applied voltage during the resistance measurements (FIG. 31) with the infrared thermal images shown in FIG. 32. As the temperature is increased from 22° C. (room temperature) to 50° C., the response/recovery time decreases from 217/650 s to 88/406 s, but the response is also reduced from 2.5% to 1.3% (to 1 ppm NO₂) (FIGS. 33-34). The accelerated response/recovery at elevated temperature results from the promoted electron transfer (crossing the potential barrier), but the accelerated desorption rate of gas molecules also results in a smaller response. This temperature-dependent behavior is consistent with the results of room-temperature NO_{X} gas sensors in the previous literature reports.

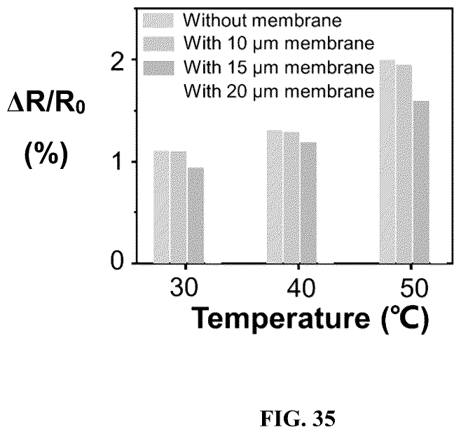

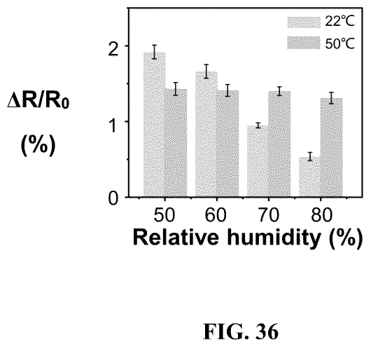

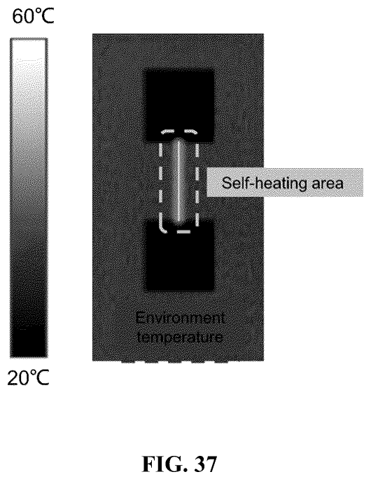

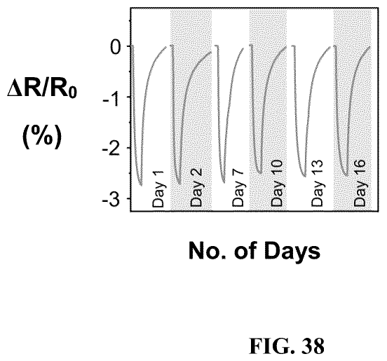

As the relative humidity (RH) level can vary in a large range in greenhouse and soil environments, we analyzed the influence of RH on the gas sensing performance of the embodiment of the sensor 100. Because of the adsorption competition between NO₂ and water molecules on the sensor surface in the high RH range, the response of the sensor to 1 ppm NO₂ decreases from 1.92% to 0.54% as the RH level is increased from 50% to 80% at room temperature (FIG. 35). However, the influence of RH on the NO₂ response can be drastically reduced when the sensor is operated at elevated temperatures (e.g., a response of 1.43/1.41/1.4/1.31% in the RH of 50/60/70/80% at 50° C.) (FIGS. 36-37). The elevated temperature in the sensing area creates thermal radiation to drive the water molecules away from the region. Additional strategies such as superhydrophobic coating can be further explored to minimize the effects of humidity. The sensor was found to be highly stable over time, as evidenced by the almost unchanged response to 1 ppm NO₂ for 16 days (FIG. 38), demonstrating a high potential for practical applications.

### Example 4: Gas Sensing Mechanisms

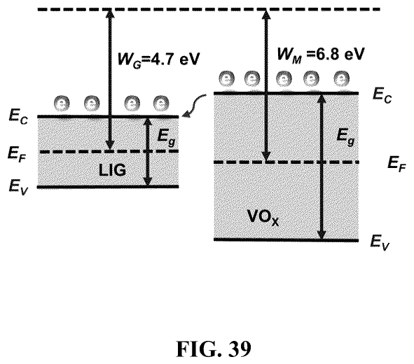

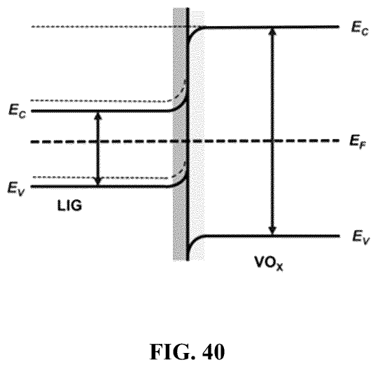

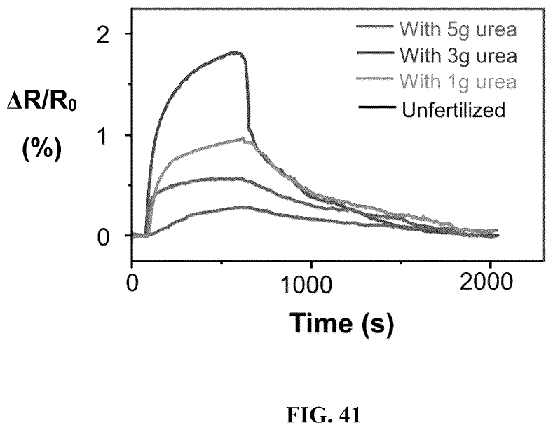

The NO_{X} adsorbed on the VO_{X}/LIG surface for the non-encapsulated sensor element of the sensor 100 was able to continuously extract electrons and extended the hole (main carriers) accumulation zone on the VO_{X}/LIG surface to lower the resistance, resulting in negative values in the relative changes. In the VO_{X}-doped LIG sensor embodiment that we evaluated, the work functions of LIG and VO_{X} are ca. 4.7 (W_{G}) and 6.8 eV (W_{M}), respectively. Due to the difference in work function, the majority charge carriers (holes and electrons) in the p-type LIG and n-type VO_{X} migrate across the heterojunction established at the interface forming a depletion layer by energy band bending (FIG. 39). Heterojunction systems have been recognized to explain the enhanced gas sensing characteristics of ZnO/rGO, ZnO/NiO, CuO/ZnO, CoO/SnO₂, PdO—ZnO, ZnFe₂O₄—ZnO in previous papers. The exposure of VO_{X}-doped LIG to NO₂ traps electrons from the conduction band and further bends the energy band (FIG. 40) to decrease the resistance. Compared with pure LIG, the VO_{X}-doped LIG shows enhanced carrier transfer, leading to higher conductivity as shown in the linear I-V curves (FIG. 41). Small changes in charge carrier concentration can also lead to great enhancement in the sensor response. The decoration of VO_{X} also decreases the initial concentration of electrons in LIG, providing a larger change in NO₂ gas adsorption and response per electron.

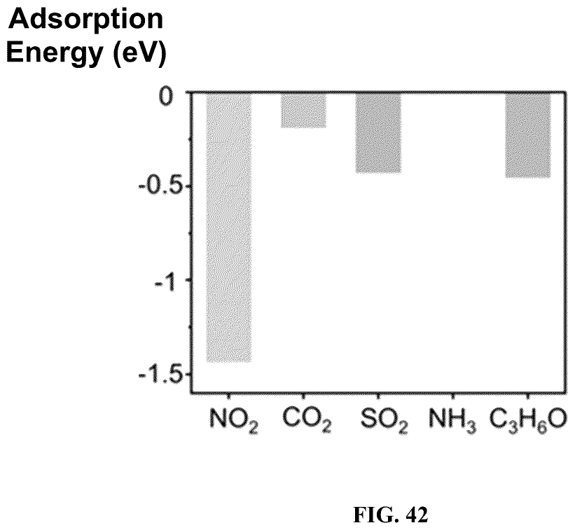

The difference in the structure and electronic properties between pristine LIG and VO_{X}-doped LIG can be revealed by the density functional theory (DFT) calculations. Compared to pristine LIG, the VO_{X}-doped LIG with heterostructures exhibits enhanced electronic levels near the Fermi level to elevate electron transfer due to the VO_{X} decoration. Deformation charge density elucidates large charge transfer between VO_{X} and LIG and the adsorption of NO₂ on the sensor surface. The adsorption energy E_{ad} of gas molecules on the sensor surface can be calculated according to Equation (2):

$${E}_{a d} = {E}_{{VO}_{X} / LIG + gas} - {E}_{{VO}_{X} / LIG} - {E}_{gas} , \qquad ( 2 )$$

where E_{VO}_{_{X}}_{/LIG} and E_{VO}_{_{X}}_{/LIG+gas} are the total energy of the system before and after the adsorption of gas molecules and E_{gas} is the energy of the isolated gas molecule. The larger negative values of E_{ad} correspond to the stronger interaction between the sensor surface and the gas molecule. The adsorption energy of −1.434 eV of NO₂ on VO_{X}-doped LIG is almost 7 times of −0.219 eV on pristine LIG, indicating improved interaction between NO₂ and VO_{X}-doped LIG. Meanwhile, the adsorption energies of other gas molecules (e.g., NH₃, SO₂, CO₂, and acetone) on VO_{X}-doped LIG are lower in magnitude than that of NO₂ (FIG. 42), which supports the highly selective detection of NO₂ over interfering gas molecules.

### Example 5: Temperature Sensing Performance

We conducted a study evaluating the temperature sensing performance based on the embodiment of the sensor as designed above in Example 1.

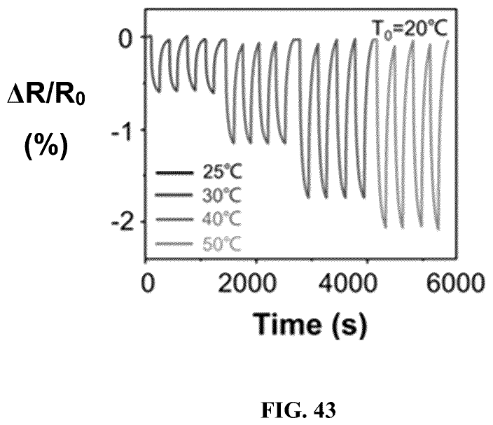

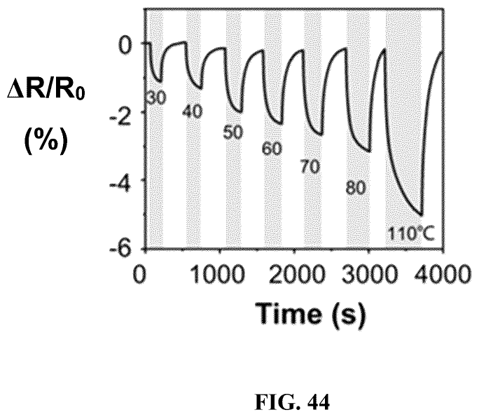

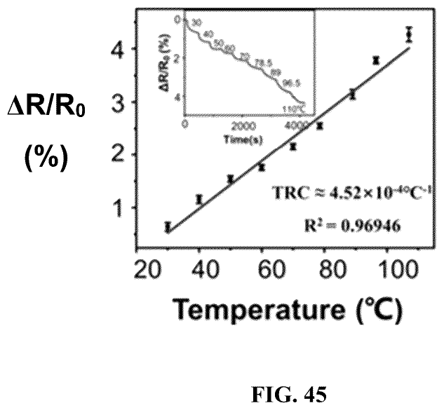

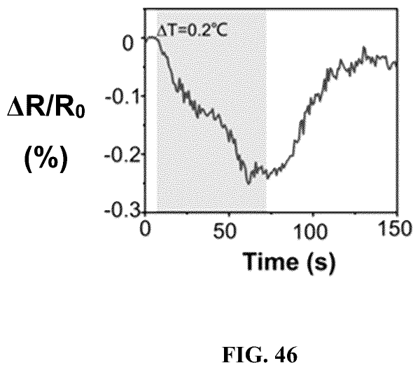

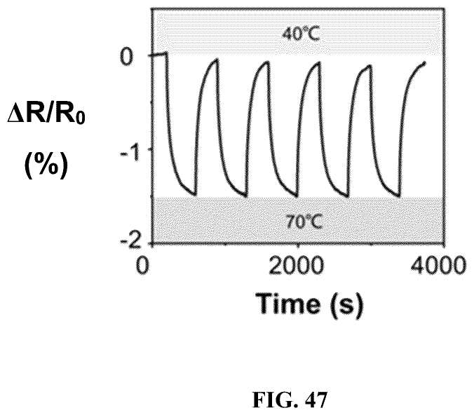

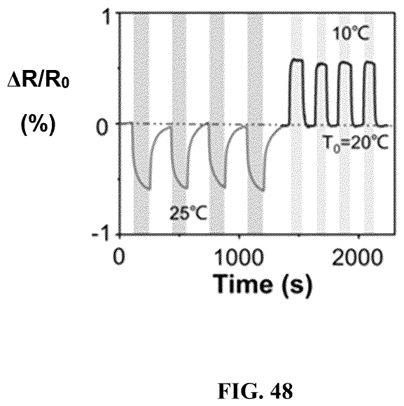

Due to the high electron mobility, superior thermal conductivity, and structural stability at high temperatures of LIG, the multi-parameter sensor shows a sensitive response with high repeatability (FIG. 43) over a wide temperature range from 30 to 110° C. (FIG. 44). The fitting of the linear calibration curve gives a negative temperature coefficient and a sensitivity of 4.52×10⁻⁴° C.⁻¹ (R²=0.97) (FIG. 45). Further increased linearity (R²=0.99) is observed in the temperature range from 30 to 50° C. (relevant for soil, human body, and infant formula temperatures). The sensor also exhibits a low limit of detection of 0.2° C. (FIG. 46) and good repeatability over heating-cooling cycles (FIGS. 47-48) to monitor both subtle and large temperature changes in practical applications.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

What is claimed is:

1. An apparatus comprising:
a first sensor comprising:
a first base layer,
a first film coated on the first base layer, wherein the first film comprises a first block copolymer carbon containing material and a first VO_{X} precursor compound, and
at least one laser-induced graphene electrode scribed on the first film, wherein the at least one electrode is doped with VO_{X} particles; and
a second sensor comprising:
a second base layer,
a second film coated on the second base layer, wherein the second film comprises a second block copolymer carbon containing material and a second VO_{X} precursor compound,
at least one laser-induced graphene electrode scribed on the second film, wherein the at least one electrode is doped with VO_{X} particles, and
a membrane configured to encapsulate the second sensor.

2. The apparatus of claim 1, wherein the first sensor is configured to collect nitrogen oxide concentration data.

3. The apparatus of claim 1, wherein the second sensor is configured to collect temperature data.

4. The apparatus of claim 1, wherein the first sensor is configured to self-heat.

5. The apparatus of claim 1, wherein the membrane is configured to block a permeation of nitrogen oxide gas molecules.

6. The apparatus of claim 1, wherein the first sensor and the second sensor are integral with each other such that the first base layer is integral with the second base layer and the second film is integral with the first film.

7. The apparatus of claim 1, wherein the first block copolymer carbon containing material and the second block copolymer carbon containing material comprise F-127-resols.

8. The apparatus of claim 2, wherein the first sensor has a detection range of 3 ppb to 5 ppm NO₂.

9. The apparatus of claim 3, wherein the second sensor has a detection range from 10-110° C.

10. A method of collecting soil data, comprising:
providing an electronic sensing device comprising:
a controller unit,
a first sensor connected to the controller unit, the first sensor comprising a base layer, a film coated on the base layer, wherein the film comprises a block copolymer carbon containing material and a VO_{X} precursor compound, and laser-induced graphene foam scribed on the film, wherein the foam is doped with VO_{X} particles;
a second sensor connected to the controller unit, the second sensor comprising a base layer, a film coated on the base layer, wherein the film comprises a block copolymer carbon containing material and a VO_{X} precursor compound, and laser-induced graphene foam scribed on the film, wherein the foam is doped with VO_{X} particles;
a non-transitory computer readable medium connected to the controller unit,
a transceiver unit connected to the controller unit, and
a power source;
collecting nitrogen oxide concentration data via the first sensor and collecting temperature data via the second sensor; and
transmitting the collected nitrogen oxide concentration data and the temperature data to an input/output device or a central computer device for evaluation of the collected nitrogen oxide concentration data and the collected temperature data.

11. The apparatus of claim 10, wherein the first sensor and the second sensor are integral with each other such that the first base layer is integral with the second base layer and the second film is integral with the first film.

12. The method of claim 10, wherein the step of transmitting the collected nitrogen oxide concentration data and the temperature data to the input/output device or the central computer device comprises:
establishing a wireless communication between the electronic device and the input/output device by positioning the input/output device in proximity to the electronic device; and
transmitting the collected nitrogen oxide concentration data and the temperature data via the transceiver unit, wherein the transceiver unit a Bluetooth transceiver unit.

13. The method of claim 12, wherein the step of stablishing the wireless communication between the electronic device and the input/output device or the central computer device comprises:
positioning the input/output device in proximity to the electronic device via a drone or vehicle.

14. The method of claim 10, wherein the transmitting the collected nitrogen oxide concentration data and the temperature data to the input/output device or the central computer device comprises
establishing a wireless communication between the electronic device and the input/output device by positioning the input/output device in proximity to the electronic device; and
transmitting the collected nitrogen oxide concentration data and the temperature data via the transceiver unit, wherein the transceiver unit a radio frequency identification transceiver unit.

15. The method of claim 14, wherein the step of establishing the wireless communication between the electronic device and the input/output device or the central computer device comprises:
positioning the input/output device in proximity to the electronic device via a drone or vehicle.

16. An apparatus comprising:
a first sensor device comprising:
a first base layer,
a first film coated on the first base layer, wherein the first film comprises a first block copolymer carbon containing material and a first VO_{X} precursor compound, and
a first set of laser-induced graphene electrodes positioned in the first base layer and/or first film to detect temperature data;
a second set of laser-induced graphene electrodes positioned in the first base layer and/or first film to detect nitrous oxide concentration data; and
a membrane positioned to encapsulate the first set of electrodes.

17. The apparatus of claim 16, wherein the first sensor is configured to self-heat.

18. The apparatus of claim 16, wherein the membrane is configured to block a permeation of nitrous oxide gas molecules.

19. The apparatus of claim 16, wherein the first block copolymer carbon containing material and the second block copolymer carbon containing material comprise F-127-resols.

20. The apparatus of claim 16, wherein the first set of electrodes and second set of electrodes are doped with VO_{X} particles.
